(19) United States
(12) Patent Application Publication
(10) Pub. No.: US 2026/0240448 A1
(43) Pub. Date: Aug. 20, 2026

(54) SYSTEMS AND METHODS FOR FOUNDATION-MODEL-GUIDED SEGMENTATION OF PELVIC MRI

(71) Applicants: Case Western Reserve University, Cleveland, OH (US); Washington University in St. Louis, St. Louis, MO (US)

(72) Inventors: Satish E. Viswanath, Beachwood, OH (US); Atreya Sridharan, Cleveland, OH (US); Thomas DeSilvio, Lyndhurst, OH (US); Brennan T. Flannery, Cleveland Heights, OH (US); Michael Kong, Cleveland, OH (US); Deepak Parakkal, St. Louis, MO (US); David Ballard, St. Louis, MO (US)

(21) Appl. No.: 19/541,531

(22) Filed: Feb. 17, 2026

Related U.S. Application Data
(60) Provisional application No. 63/759,348, filed on Feb. 17, 2025.

(51) Int. Cl. A61B 5/055; G06T 7/00; G06V 10/764

(52) U.S. Cl. CPC A61B 5/055; G06T 7/0012; G06V 10/764

(57) ABSTRACT

In an embodiment, a system includes electronic memory configured to store pelvic magnetic resonance imaging (MRI) data and one or more processors. The one or more processors are configured to operate a first deep learning segmentation model upon the pelvic MRI data to generate an initial localization of a plurality of target structures in the pelvic MRI data. The one or more processors operate the first deep learning segmentation model to generate a probability map corresponding to the plurality of target structures. Enhanced imaging data is generated using the initial localization, the probability map, and the pelvic MRI data. The one or more processors operate a second deep learning segmentation model upon the enhanced imaging data to generate segmented imaging data identifying locations of the plurality of target structures.

### REFERENCE TO RELATED APPLICATION

This application claims priority to and the benefit of U.S. Provisional Patent Application No. 63/759,348, filed Feb. 17, 2025, the entire disclosure of which is incorporated by reference herein in its entirety.

### GOVERNMENT SUPPORT CLAUSE

This invention was made with government support under CA280981 awarded by the National Institutes of Health. The government has certain rights in the invention.

### TECHNICAL FIELD

The present disclosure relates to medical image processing and, more particularly, to systems and methods that integrate a foundation segmentation model and a self-configuring segmentation model for automated segmentation in pelvic magnetic resonance imaging (MRI).

### BACKGROUND

Crohn's disease is a chronic inflammatory bowel disease (IBD) that affects the lining of the digestive tract. Over time, it can cause swelling of digest tract tissue, which can lead to symptoms such as abdominal pain, diarrhea, fatigue, weight loss, and malnutrition. Perianal fistulas are a common complication associated with Crohn's disease and frequently require interventional guidance and surgical planning.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a block diagram of some embodiments of an integrated segmentation system to automatically segment digitized imaging data to identify a target structure.

FIG. 2 illustrates a flow diagram of some embodiments of a method of training a foundation segmentation model using boundary box prompts derived from manual annotations.

FIG. 3 illustrates a flow diagram of some embodiments of a method of training a self-configuring segmentation model using enhanced imaging data generated using a probability map produced by the foundation segmentation model.

FIG. 4 illustrates a flow diagram of some embodiments of a method of automatically segmenting digitized imaging data at runtime using a trained foundation segmentation model and a trained self-configuring segmentation model.

FIG. 5 illustrates a diagram of some embodiments of probability-map weighting for generating enhanced imaging data.

FIG. 6 illustrates a diagram of some embodiments of boundary box prompting and random prompt sampling for a foundation segmentation model.

FIG. 7 illustrates an example schematic representation of some embodiments of a foundation segmentation model including an image encoder, a prompt encoder, and a mask decoder.

FIG. 8 illustrates an example pipeline for segmentation of MRI scans including image quality evaluation, manual annotation, boundary box prompting, foundation-model localization, and self-configuring model segmentation.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding. Unless the context clearly indicates otherwise, the singular forms “a,” “an,” and “the” include the plural forms as well. In addition, the term “or” is intended to mean “and/or,” unless the context clearly indicates otherwise. Features, elements, steps, and/or operations described with reference to one figure may be combined with, substituted for, or used in connection with features, elements, steps, and/or operations described with reference to other figures where appropriate.

A significant proportion of Crohn's disease cases develop perianal fistulizing Crohn's Disease (CD-PAF), a condition characterized by the formation of perianal fistulas. These fistulas are caused by chronic inflammation, which damages the tissue in the anal and rectal area, forming abnormal tunnels between the anus and the surrounding skin. A majority of CD-PAF patients require interventional treatment such as surgery. Interventional guidance for CD-PAF requires clinicians to be able to accurately visualize anorectal structures most commonly associated with CD-PAF. Such visualization can enable targeted surgical management and evaluation of fistula response to therapy.

Pelvic magnetic resonance imaging (MRI) is routinely used for determining localization of perianal fistula tracts and their relationship to surrounding anorectal anatomy. However, inter-reader variability can be significant (e.g., 0.6-0.8, which can be lower when evaluating specific aspects of fistula appearance on MRI), due to diverse presentations on MRI, including variability in shape, size, signal intensity, and the presence of multiple tortuous tracts. As a result, improved automating tools for accurate and consistent identification and visualization of the anal sphincter complex (including internal and external sphincter muscles) and perianal fistulae are desirable to support clinical decision-making.

Deep learning segmentation models can automate structure delineation, but self-configuring models (e.g., nnU-Net) are typically dependent on expert annotations and can be sensitive to domain shift across scanners and institutions. Foundation models provide prompt-based segmentation across a wide range of structures, but foundation-model outputs may be insufficiently precise for specialized pelvic anatomy and thin, branching fistula tracts.

Accordingly, improved techniques are desirable that combine robust localization capability of foundation models with structure-specific optimization of self-configuring models for pelvic MRI segmentation in CD-PAF. In some embodiments, a foundation model is trained and later deployed to generate not only an initial bounded input image, but also generate a weighted probability map to enhance spatial contextualization of the initial bounded input image. The enhanced bounded input image is fed as an input to a trained self-configuring model to provide for structure-specific feature optimization. Various embodiments of the present disclosure will now be discussed in detail with reference to the accompanying figures. These figures depict example configurations of systems and methods that use integrated deep learning models to perform automatic segmentation of fistula tracts and anorectal structures (e.g., the internal and external sphincter muscles) of pelvic MRI data, as described herein.

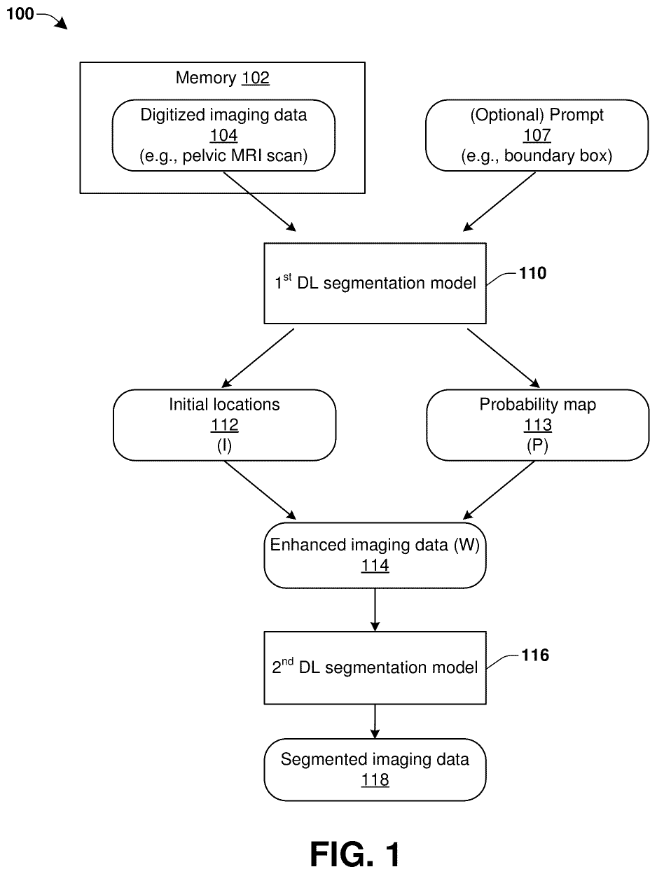

FIG. 1 illustrates a block diagram of an integrated segmentation system 100 to automatically segment digitized imaging data to identify a plurality of target structures in accordance with some embodiments. The plurality of target structures includes an anatomical structure and a pathological region. In an embodiment, the anatomical structure includes an internal sphincter muscle or an external sphincter muscle, and the pathological region includes a fistula tract. The disclosed integration addresses pelvic MRI characteristics that differ from many other segmentation settings, such as for tumor-style approaches. A fistula tract can be thin, branching, and tortuous with variable signal intensity, and can traverse tissue planes near an anal sphincter complex. Small geometric errors can materially change clinical interpretation of whether a tract crosses, penetrates, or abuts a sphincter structure.

As shown in FIG. 1, the system 100 comprises electronic memory 102 (e.g., solid state memory, SRAM (static random-access memory), DRAM (dynamic random-access memory), and/or the like) that is configured to receive or store digitized imaging data 104. In an embodiment, the digitized imaging data 104 includes digitized images, such as a pelvic MRI, from a patient that has perianal fistulizing Crohn's disease. The system 100 receives digitized imaging data 104 and operates a first deep learning segmentation model 110 to generate an initial localization 112 and a probability map 113. The system 100 generates enhanced imaging data 114 using the initial localization 112 and the probability map 113, and then operates a second deep learning segmentation model 116 to generate segmented imaging data 118. The segmented imaging data 118 may then be used for a clinical workflow output.

In an embodiment, the digitized imaging data 104 is pre-processed prior to model operation, and the pre-processing includes normalization, resampling, cropping to a region of interest, denoising, motion correction, bias-field correction, artifact reduction, and field-of-view centering. The digitized imaging data 104 may comprise one or more CT images, MRI images, contrast CT images, and/or the like. In an embodiment, the digitized imaging data 104 includes MRI data. In an embodiment, the digitized imaging data 104 includes pelvic MRI data such as a a T2-weighted pelvic MRI scan. The MRI scan may be acquired from a patient at any point in their clinical evaluation, such as taken before or after treatment.

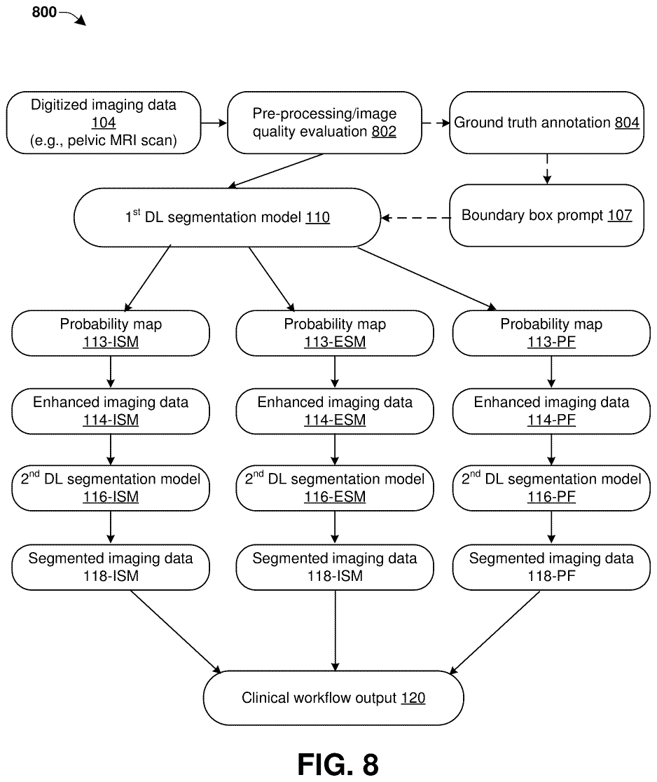

As explained in more details associated with FIG. 8, in an embodiment, a 3D pelvic MRI volume is processed as a plurality of 2D sections. Outputs for the 2D sections are reconstructed to form a 3D probability map or a 3D initial localization.

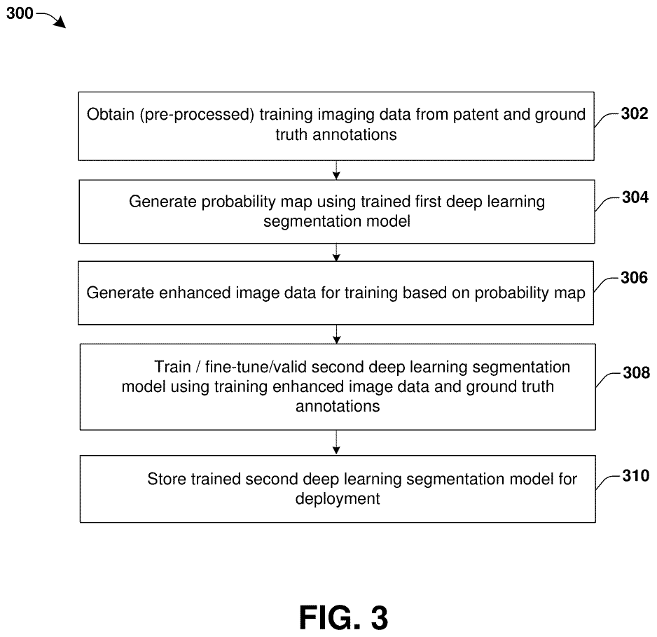

In an embodiment, a prompt 107, is optionally provided to the first deep learning segmentation model 110 for generating the initial localization 112 or the probability map 113. The prompt 107 provides spatial guidance to the first deep learning segmentation model 110. In an embodiment, the prompt 107 includes a boundary box. During training, the boundary box may be generated from a manual annotation and may be perturbed to perform random prompt sampling (will be discuss further below associated with FIG. 3). During deployment, the boundary box may be provided by a user interface or is generated by a detector or a heuristic region-of-interest routine.

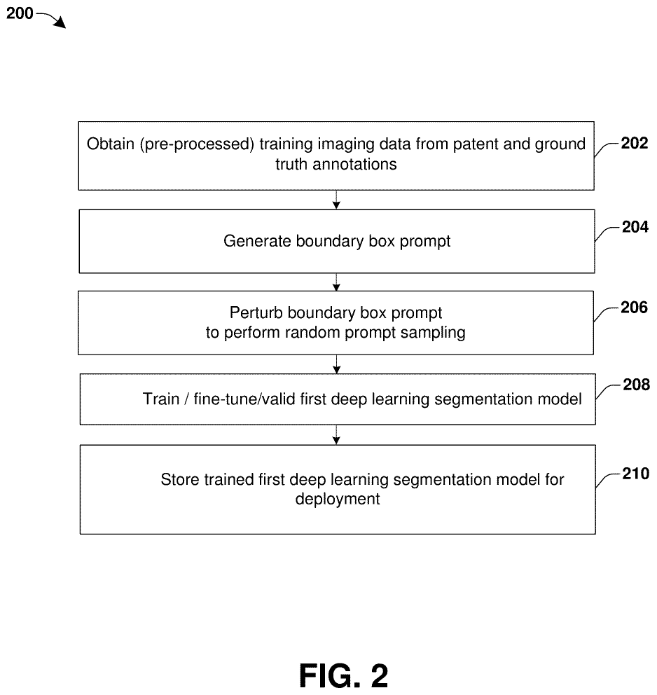

The first deep learning segmentation model 110 is configured to operate on the digitized imaging data 104 and optionally also the prompt 107 to generate the initial localization 112 and the probability map 113. In an embodiment, the first deep learning segmentation model 110 be or be comprised of a foundation segmentation model configured for prompt-based segmentation. In an embodiment, the first deep learning segmentation model 110 is a Medical Segment Anything Model (MedSAM). The probability map 113 encodes spatial likelihood information for a target structure and is aligned with the digitized imaging data 104. As discussed in greater detail later associated with FIG. 2, the first deep learning segmentation model 110 may be trained and validated before deployment.

In an embodiment, the enhanced imaging data 114 is generated using the probability map 113 to emphasize the initial localization 112. For example, enhanced imaging data is generated as a weighted image W=I*P, where I denotes the digitized imaging data and P denotes the probability map. As used herein, “initial localization” may refer to an approximate spatial indication of a structure of interest, such as a coarse segmentation mask or a region-of-interest. The initial localization 112 may be represented by a coarse mask or a region-of-interest output directly by the first deep learning segmentation model 110 as shown in FIG. 1, or derived from the probability map 113. As used herein, “probability map” may refer to an image or volume aligned array of values that encode a spatial likelihood distribution for a structure of interest.

In an embodiment, the second deep learning segmentation model 116 operates on the enhanced imaging data 114 to generate the segmented imaging data 118. In an embodiment, the second deep learning segmentation model 116 is a self-configuring segmentation model. The second deep learning segmentation model 116 may be or be comprised of a self-configuring model. In an embodiment, the second deep learning segmentation model 116 is a U-net model, a nnU-net model, and/or the like.

As discussed in greater detail later associated with FIG. 3, the second deep learning segmentation model 116 may be trained and validated before deployment.

In an embodiment, the second deep learning segmentation model 116 may segment the plurality of target structures individually. In an alternative embodiment, the second deep learning segmentation model 116 may be configured as a multi-class self-configuring model to jointly segment the the plurality of target structures.

In an embodiment, the segmented imaging data 118 is displayed as an overlay on the digitized imaging data 104 on a workstation (not shown in FIG. 1) to facilitate surgical planning or guidance. In an embodiment, the segmented imaging data 118 is exported as a DICOM segmentation object for storage in a picture archiving and communication system (PACS) or used to generate a structured report.

The first deep learning segmentation model 110 and the second deep learning segmentation model 116 may be run on one or more processors (e.g., a central processing unit including one or more transistor devices configured to operate computer code to achieve a result, a microcontroller, or the like, a GPU, and/or the like).

FIG. 2 illustrates a flow diagram corresponding to some embodiments of a method 200 of training or validating a foundation segmentation model, such as the first deep learning segmentation model 110 of FIG. 1, using boundary box prompts derived from manual annotations, as an example.

At act 202, training imaging data is obtained together with a ground truth annotation for a target structure. In an embodiment, the ground truth annotation is an expert manual annotation or a segmentation mask for the target structure. In an embodiment, the target structure includes an anatomical structure, such as an internal sphincter muscle (ISM) or an external sphincter muscle (ESM), or a pathological region, such as a fistula tract (FIS) of a patient. In an embodiment, the training imaging data comprises MRI data, such as T2-weighted MRI scans, which have been normalized, resampled, cropped to a region of interest, and/or otherwise pre-processed. The MRI data may be acquired from a patient at any point in their clinical evaluation, such as taken before or after treatment.

At act 204, in an embodiment, a boundary box prompt is supplied to the foundation segmentation model to indicate an approximate region for the structure of interest. The boundary box prompt may be generated from the manual annotation. In an embodiment, generating the boundary box prompt includes computing a bounding box that encloses the manual annotation in a 2D section and optionally expanding the bounding box by a margin.

At act 206, in an embodiment, the boundary box prompt is perturbed to perform random prompt sampling. In an embodiment, random prompt sampling includes randomly shifting, scaling, and/or expanding the annotation-derived boundary box to generate multiple plausible prompts for the same target structure, and training the foundation segmentation model so that its output remains stable across prompt variations in the boundary box prompt.

At act 208, the foundation segmentation model is trained, fine-tuned, and validated using the training imaging data and the boundary box prompts, including the perturbed prompts. In an embodiment, training includes generating image embeddings and prompt embeddings (see FIG. 10), generating a predicted segmentation, computing a segmentation loss relative to the ground truth annotation, and updating model parameters using backpropagation. In an embodiment, the segmentation loss includes a combination of Dice loss and focal loss to improve robustness for thin and irregular structures.

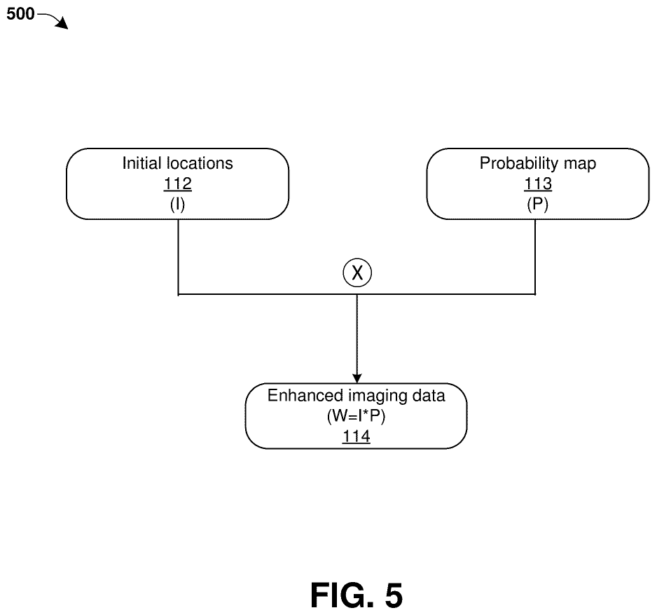

At act 210, a trained first deep learning segmentation model is stored and ready for deployment. In an embodiment, a model checkpoint and one or more validation metrics are stored, and a checkpoint corresponding to a selected validation performance is designated for deployment. When executed, based on input image data, the trained first deep learning segmentation model may be used as a foundation segmentation model that outputs an initial localization (e.g., a coarse segmentation mask or a region-of-interest indication) and a probability map aligned with the input image. In an embodiment, the probability map comprises pre-softmax logits or other unnormalized likelihood values that preserve spatial likelihood information at structure boundaries, which is later used to generate enhanced imaging data 114 (see FIGS. 3 and 5).

FIG. 3 illustrates a flow diagram corresponding to some embodiments of a method 300 of training and validating a self-configuring segmentation model, such as the second deep learning segmentation model 116 of FIG. 1, using training enhanced imaging data generated based on a probability map produced by a trained first deep learning segmentation model.

At act 302, training imaging data is obtained together with a ground truth annotation for a target structure. In an embodiment, the training imaging data comprises pelvic magnetic resonance imaging (MRI) data, such as T2-weighted MRI scans, that have been normalized, resampled, cropped to a region of interest, and/or otherwise pre-processed. The MRI scans may be acquired from a patient at any point in their clinical evaluation, such as taken before or after treatment. In an embodiment, the manual annotation provides ground truth segmentation for the target structure. For example, in an embodiment, the target structure includes an anatomical structure such as an internal sphincter muscle (ISM) or an external sphincter muscle (ESM), and the target structure further includes a pathological region such as a fistula tract (FIS).

At act 304, a trained first deep learning segmentation model, such as the trained foundation segmentation model of FIG. 2, is executed on the training imaging data to generate a probability map for the target structure. In an embodiment, the trained first deep learning segmentation model is used as a foundation segmentation model that accepts a boundary box prompt and outputs a probability map aligned with the training imaging data. In an embodiment, the probability map comprises pre-softmax logits or other unnormalized likelihood values that encode spatial likelihood information for the target structure.

At act 306, training enhanced image data is generated based on the probability map. In an embodiment, generating the training enhanced image data comprises probability-map weighting to generate a weighted image W=I×P, where I denotes the training imaging data and P denotes the probability map, and where the multiplication is performed element-wise to emphasize pixels or voxels associated with the target structure while preserving surrounding context.

At act 308, the second deep learning segmentation model is trained, fine-tuned, and validated using the training enhanced image data and the manual annotation as ground truth. In an embodiment, the second deep learning segmentation model is a self-configuring model comprising a U-net architecture, such as an nnU-Net model, that configures one or more training settings based on the training dataset. In an embodiment, training includes computing a loss between a predicted segmentation and the manual annotation and updating model parameters using backpropagation.

In an embodiment, the second deep learning segmentation model includes a plurality of per-structure models, including a first model configured to segment ISM, a second model configured to segment ESM, and a third model configured to segment FIS, each trained using corresponding training enhanced image data and corresponding ground truth manual annotations. In another embodiment, a multi-class second deep learning segmentation model is trained to jointly segment a plurality of target structures using corresponding multi-class ground truth annotations.

At act 310, a trained second deep learning segmentation model is stored and ready for deployment. In an embodiment, a model checkpoint and one or more validation metrics are stored, and a checkpoint corresponding to a selected validation performance is designated for deployment. When executed at runtime, the trained second deep learning segmentation model is configured to generate segmented imaging data identifying a location of the target structure or segmentation of various target structures based on enhanced imaging data.

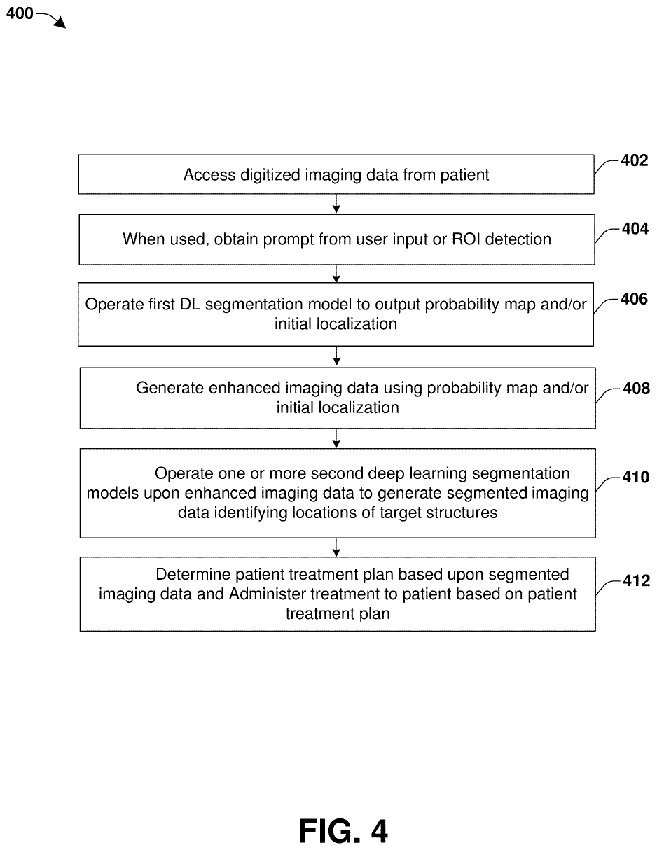

FIG. 4 illustrates a flow diagram corresponding to some embodiments of a method 400 of automatically segmenting digitized imaging data at runtime using a trained foundation segmentation model and a trained self-configuring segmentation model. In an embodiment, the method 400 is performed on pelvic MRI data of a patient to identify a target structure such as ISM, ESM, and/or FIS.

At act 402, pelvic MRI data is obtained. In an embodiment, the pelvic MRI data comprises a T2-weighted MRI scan that has been normalized and cropped to a pelvic region of interest. The MRI scan may be acquired from a patient at any point in their clinical evaluation, such as taken before or after treatment.

At act 404, optionally, a prompt is obtained. In an embodiment, the prompt includes a boundary box generated from user input (e.g., a clinician selecting a region of interest), or generated automatically using a detector or a heuristic localization routine, for example.

At act 406, a first deep learning segmentation model, such as a trained foundation segmentation model, is operated using the pelvic MRI data and, when present, the prompt. The foundation segmentation model may output a probability map (P) for the target structure and/or an initial localization (I).

At act 408, enhanced imaging data (W) is generated using probability-map weighting, such as W=I*P.

At act 410, the trained self-configuring segmentation model is operated on the enhanced imaging data to generate segmented imaging data that identifies the location of the target structure, such as fistula and one or more anorectal structures.

At act 412, a clinical workflow output may be provided using the segmented imaging data. In an embodiment, the segmented imaging data is displayed as an overlay on the pelvic MRI data for surgical planning or interventional guidance, exported as a DICOM segmentation object for storage in a picture archiving and communication system (PACS), and/or used to generate a structured report. A patient treatment plan may be determined based upon the segmented imaging data. In some embodiments, the patient treatment plan may be surgical treatment of perianal fistula tracts. A treatment may be administered to the patient based on the patient treatment plan.

While the disclosed method 400 are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

FIG. 5 illustrates a diagram 500 of some embodiments of probability-map weighting for generating enhanced imaging data, such as the enhanced imaging data 114 as discussed in FIGS. 1-4. As shown, imaging data I and a probability map P are combined to generate enhanced imaging data W. In an embodiment, W is generated by an element-wise multiplication W=I*P so that pixel or voxel intensities associated with the target structure are emphasized while preserving underlying image context.

In an embodiment, the probability map P comprises pre-softmax logits or other unnormalized likelihood values produced by the first deep learning segmentation model, thereby preserving spatial likelihood information (including boundary-related likelihood information) for the target structure. The imaging data I may be derived from scanning data after one or more pre-processing operations, such as normalization, resampling, cropping to a region of interest, denoising, and/or artifact reduction. In an embodiment, the imaging data I comprises scan-derived imaging data generated based on an output of the first deep learning segmentation model, such as a region-of-interest cropped image generated using the initial localization 112.

In an embodiment, the imaging data I and the enhanced imaging data W comprise a plurality of 2D sections (e.g., slices) corresponding to a 3D medical image volume, and the enhanced imaging data W is used to train or operate the second deep learning segmentation model to generate a 3D segmentation for the target structure. In another embodiment, the enhanced imaging data W is generated for a plurality of target structures. For example, in an embodiment, a first enhanced imaging dataset is generated for an anatomical structure (e.g., ISM or ESM) and a second enhanced imaging dataset is generated for a pathological region (e.g., FIS), and the enhanced imaging datasets are used to train or operate per-structure segmentation models. In another embodiment, the enhanced imaging data W is generated for multiple target structures and used as input to a multi-class segmentation model configured to output a segmentation for the multiple target structures.

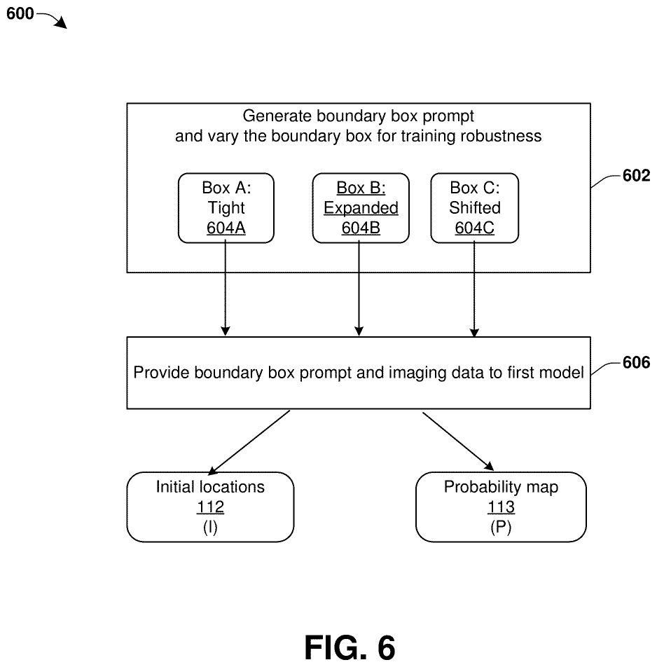

FIG. 6 illustrates a diagram 600 of some embodiments of boundary box prompting and random prompt sampling for a foundation segmentation model, such as the first deep learning segmentation model 110 of FIG. 1.

At act 602, and varied to improve training robustness. In an embodiment, the boundary box prompt is derived from a manual annotation and indicates an approximate region corresponding to a target structure in digitized imaging data. In an embodiment, varying the boundary box prompt includes generating a plurality of boundary box prompts for the same target structure, such as a tight box 604A, an expanded box 604B, and a shifted box 604C, as example instances/variations of boundary box prompt 107 in FIG. 1, for example.

At act 606, in an embodiment, the boundary box prompt and the digitized imaging data are provided to the first deep learning segmentation model. In an embodiment, the first deep learning segmentation model generates an initial localization 112 and a probability map 113 based on the digitized imaging data and the boundary box prompt. In an embodiment, training the first deep learning segmentation model using varied boundary box prompts causes the initial localization 112 and the probability map 113 to remain stable across variations in the boundary box prompt.

In an embodiment, the initial localization 112 includes a coarse segmentation mask and/or a region-of-interest indication for the target structure. In an embodiment, the probability map 113 includes pre-softmax logits or other unnormalized likelihood values aligned with the digitized imaging data. The probability map 113 may later be used to generate enhanced imaging data (e.g., by probability-map weighting), as described in connection with FIGS. 3 and 5.

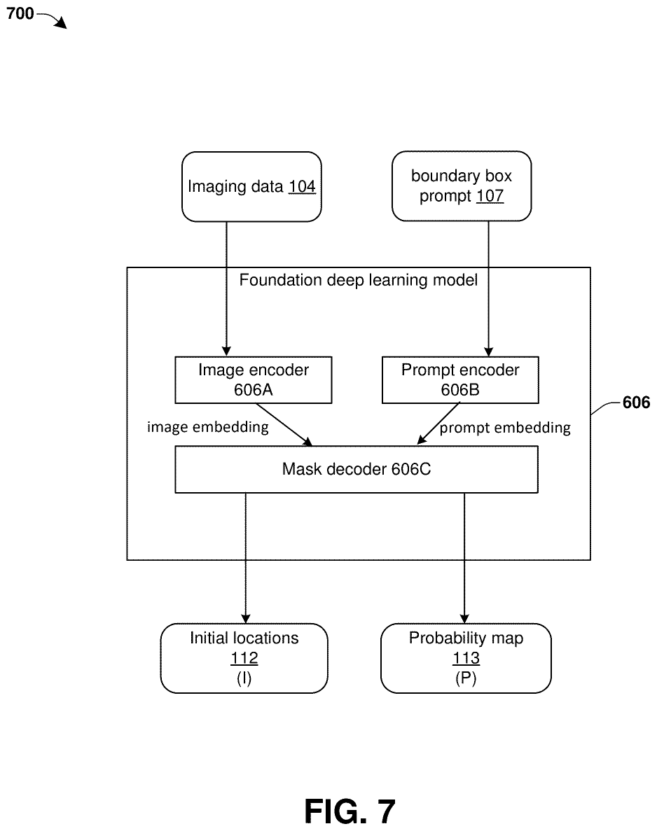

FIG. 7 illustrates a diagram 700 of some embodiments of a foundation segmentation model configured to accept digitized imaging data 104 and a boundary box prompt 107 and to output an initial localization 112 and a probability map 113. In an embodiment, FIG. 7 provides an example implementation of the first deep learning segmentation model 110 described in connection with FIGS. 1-6.

As shown in FIG. 7, digitized imaging data 104 is provided to an image encoder 606A. In an embodiment, the image encoder 606A is configured to process the digitized imaging data 104 to generate an image embedding. A boundary box prompt 107 is provided to a prompt encoder 606B. In an embodiment, the prompt encoder 606B is configured to process the boundary box prompt 107 to generate a prompt embedding. In an embodiment, the boundary box prompt 107 includes an approximate region corresponding to a target structure, and, in an embodiment, the boundary box prompt 107 includes a varied instance such as the example variations shown in FIG. 6 (e.g., 604A-604C).

The image embedding and the prompt embedding are provided to a mask decoder 606C. In an embodiment, the mask decoder 606C is configured to generate an initial localization 112 and a probability map 113 for the target structure based on the image embedding and the prompt embedding. In an embodiment, the initial localization 112 includes a coarse segmentation mask and/or a region-of-interest indication, and the probability map 113 includes pre-softmax logits or other unnormalized likelihood values that encode spatial likelihood information for the target structure.

In an embodiment, the initial localization 112 and the probability map 113 are generated at runtime using a trained foundation segmentation model (see FIG. 4). In an embodiment, the probability map 113 is used to generate enhanced imaging data by probability-map weighting, such as described in connection with FIG. 5, and the enhanced imaging data is provided to a second deep learning segmentation model for refined segmentation.

FIG. 8 illustrates an example pipeline 800 for automatically segmenting pelvic magnetic resonance imaging (MRI) data to identify a plurality of target structures using an integrated foundation-model-guided segmentation approach. In an embodiment, the plurality of target structures includes an internal sphincter muscle (ISM), an external sphincter muscle (ESM), and a perianal fistula (PF).

As shown in FIG. 8, digitized imaging data 104 (e.g., pelvic MRI data such as T2-weighted MRI scans) is obtained and optionally subjected to image quality evaluation and pre-processing 802. The MRI scan may be acquired from a patient at any point in their clinical evaluation, such as taken before or after treatment. In an embodiment, the pre-processing 802 includes normalizing intensities, resampling to a target spacing, cropping to a region of interest, and excluding images having an inaccurately centered field-of-view, noise, and/or artifacts.

A boundary box prompt 107 is obtained or generated based a ground truth annotation, and then fed to a first deep learning segmentation model 110, together with the digitized imaging data 104 after pre-processing. In an embodiment, the boundary box prompt 107 indicates an approximate region corresponding to a target structure and may be derived from a manual annotation or another localization technique. In an embodiment, the first deep learning segmentation model 110 comprises a foundation segmentation model configured to accept spatial prompting and to output spatial likelihood information for the target structure.

In an embodiment, the first deep learning segmentation model 110 generates, for each target structure, an initial localization and a corresponding probability map aligned with the digitized imaging data 104. In an embodiment, the probability map comprises pre-softmax logits or other unnormalized likelihood values. In the illustrated example, the first deep learning segmentation model 110 generates an ISM probability map 113-ISM, an ESM probability map 113-ESM, and a PF probability map 113-PF, thereby forming three parallel lanes for ISM, ESM, and PF, respectively.

In an embodiment, enhanced imaging data is generated within each lane using probability-map weighting based on the corresponding probability map and the digitized imaging data 104. In an embodiment, probability-map weighting comprises an element-wise multiplication W=I×P to generate an enhanced imaging data representation that emphasizes image regions associated with the target structure while preserving underlying anatomical context. In the illustrated example, ISM-enhanced imaging data 114-ISM is generated based on 113-ISM, ESM-enhanced imaging data 114-ESM is generated based on 113-ESM, and PF-enhanced imaging data 114-PF is generated based on 113-PF.

In an embodiment, each enhanced imaging data stream is provided to a corresponding second deep learning segmentation model to generate segmented imaging data for the target structure. In the illustrated example, a second deep learning segmentation model 116-ISM processes 114-ISM to generate 118-ISM, a second deep learning segmentation model 116-ESM processes 114-ESM to generate 118-ESM, and a second deep learning segmentation model 116-PF processes 114-PF to generate 118-PF.

In an embodiment, the segmented imaging data is used to generate a clinical workflow output 120, such as an overlay visualization, a quantitative measurement, and/or a report or planning output indicating a relationship between a pathological region (e.g., PF) and anatomical structures (e.g., ISM and ESM). FIG. 8 thereby illustrates, in a single view, an example implementation of the disclosed integrated foundation-model-guided segmentation approach using three parallel lanes for ISM, ESM, and PF.
Example 1. A system includes electronic memory configured to store pelvic magnetic resonance imaging (MRI) data. The system further includes one or more processors configured to operate a first deep learning segmentation model upon the pelvic MRI data to generate an initial localization of a plurality of target structures in the pelvic MRI data. The one or more processors are further configured to operate the first deep learning segmentation model to generate a probability map corresponding to the plurality of target structures. The one or more processors are further configured to generate enhanced imaging data using the initial localization, the probability map, and the pelvic MRI data. The one or more processors are further configured to operate a second deep learning segmentation model upon the enhanced imaging data to generate segmented imaging data identifying locations of the plurality of target structures.
Example 2. The system of example 1, where generating the enhanced imaging data includes multiplying the pelvic MRI data by the probability map to generate a weighted image.
Example 3. The system of one of examples 1 or 2, where a first target structure of the plurality of target structures is an anatomical structure and a second target structure of the plurality of target structures is a pathological region.
Example 4. The system of one of examples 1 to 3, where the anatomical structure includes an internal sphincter muscle or an external sphincter muscle, and the pathological region includes a fistula tract.
Example 5. The system of one of examples 1 to 4, where the pelvic MRI data includes a T2-weighted MRI scan.
Example 6. The system of one of examples 1 to 5, where the first deep learning segmentation model is a foundation segmentation model, and where the second deep learning segmentation model is a self-configuring segmentation model, and where the foundation segmentation model includes a Medical Segment Anything Model (MedSAM) and the self-configuring segmentation model includes an nnU-Net model.
Example 7. The system of one of examples 1 to 6, where the one or more processors are further configured to display an overlay of the segmented imaging data on the pelvic MRI data.
Example 8. A method, including: accessing pelvic magnetic resonance imaging (MRI) data of a patient. Operating a first deep learning segmentation model upon the pelvic MRI data to generate an initial localization of a plurality of target structures in the pelvic MRI data. Operating the first deep learning segmentation model to generate a probability map corresponding to the plurality of target structures. Generating enhanced imaging data using the initial localization, the probability map, and the pelvic MRI data. Operating a second deep learning segmentation model upon the enhanced imaging data to generate segmented imaging data identifying locations of the plurality of target structures. And providing an output for a clinical workflow based upon the segmented imaging data.
Example 9. The system of example 8, where generating the enhanced imaging data includes multiplying the pelvic MRI data by the probability map to generate a weighted image.
Example 10. The system of one of examples 8 or 9, where a first target structure of the plurality of target structures is an anatomical structure and a second target structure of the plurality of target structures is a pathological region.
Example 11. The system of one of examples 8 to 10, where the anatomical structure includes an internal sphincter muscle or an external sphincter muscle, and the pathological region includes a fistula tract.
Example 12. The system of one of examples 8 to 11, further including generating a boundary box for a target structure of the plurality of target structures, and providing the boundary box as a prompt to the first deep learning segmentation model.
Example 13. The system of one of examples 8 to 12, where the second deep learning segmentation model is a self-configuring segmentation model.
Example 14. A non-transitory computer-readable medium storing computer-executable instructions that, when executed, cause a processor to perform operations, including accessing pelvic magnetic resonance imaging (MRI) data of a patient. The operations include operating a first deep learning segmentation model upon the pelvic MRI data to generate an initial localization of a plurality of target structures in the pelvic MRI data. The operations further include operating the first deep learning segmentation model to generate a probability map corresponding to the plurality of target structures. The operations include generating enhanced imaging data using the initial localization, the probability map, and the pelvic MRI data. The operations further include operating a second deep learning segmentation model upon the enhanced imaging data to generate segmented imaging data identifying locations of the plurality of target structures.
Example 15. The system of example 14, where generating the enhanced imaging data includes multiplying the pelvic MRI data by the probability map to generate a weighted image.
Example 16. The system of one of examples 14 or 15, where a first target structure of the plurality of target structures is an anatomical structure and a second target structure of the plurality of target structures is a pathological region.
Example 17. The system of one of examples 14 to 16, where the second deep learning segmentation model is a self-configuring segmentation model.
Example 18. The system of one of examples 14 to 17, where the second deep learning segmentation model includes a plurality of second deep learning segmentation models that are respectively configured to segment different target structures of the plurality of target structures.
Example 19. The system of one of examples 14 to 18, where the second deep learning segmentation model includes a multi-class segmentation model configured to segment the plurality of target structures.
Example 20. The system of one of examples 14 to 19, where the operations further include evaluating the pelvic MRI data for imaging quality, excluding an image having an inaccurately centered field-of-view, noise, or artifact, and exporting the segmented imaging data as a DICOM segmentation object for storage in a picture archiving and communication system.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device. In an embodiment, aspects of the present disclosure are implemented as a method performed by one or more processors, a system including one or more processors and a memory storing instructions, and a non-transitory computer-readable medium storing instructions that, when executed, cause performance of operations described herein.

Although the embodiments are discussed primarily in the context of pelvic magnetic resonance imaging (MRI) for segmenting a pathological region relative to anorectal anatomy, the disclosed systems and methods are not limited to that clinical context. In an embodiment, the techniques are applied to MRI data of other anatomical regions by selecting an imaging protocol and training data appropriate for a region of interest while maintaining the integrated workflow in which a foundation segmentation model generates a probability map that is used to form enhanced imaging data for a second segmentation model.

Embodiments discussed herein relate to training and/or employing machine learning models (e.g., unsupervised (e.g., clustering) or supervised (e.g., classifiers, etc.) models) to determine a medical prediction based on a combination of radiomic features and deep learning, based at least in part on features of medical imaging scans (e.g., MRI, CT, etc.) that are not perceivable by the human eye, and involve computation that cannot be practically performed in the human mind. As one example, machine learning classifiers and/or deep learning models as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

What is claimed is:

1. A system, comprising:
electronic memory configured to store pelvic magnetic resonance imaging (MRI) data; and
one or more processors configured to:
operate a first deep learning segmentation model upon the pelvic MRI data to generate an initial localization of a plurality of target structures in the pelvic MRI data;
operate the first deep learning segmentation model to generate a probability map corresponding to the plurality of target structures;
generate enhanced imaging data using the initial localization, the probability map, and the pelvic MRI data; and
operate a second deep learning segmentation model upon the enhanced imaging data to generate segmented imaging data identifying locations of the plurality of target structures.

2. The system of claim 1, wherein generating the enhanced imaging data comprises multiplying the pelvic MRI data by the probability map to generate a weighted image.

3. The system of claim 1, wherein a first target structure of the plurality of target structures is an anatomical structure and a second target structure of the plurality of target structures is a pathological region.

4. The system of claim 3, wherein the anatomical structure comprises an internal sphincter muscle or an external sphincter muscle, and the pathological region comprises a fistula tract.

5. The system of claim 1, wherein the pelvic MRI data comprises a T2-weighted MRI scan.

6. The system of claim 1, wherein the first deep learning segmentation model is a foundation segmentation model, and wherein the second deep learning segmentation model is a self-configuring segmentation model, and wherein the foundation segmentation model comprises a Medical Segment Anything Model (MedSAM) and the self-configuring segmentation model comprises an nnU-Net model.

7. The system of claim 1, wherein the one or more processors are further configured to display an overlay of the segmented imaging data on the pelvic MRI data.

8. A method, comprising:
accessing pelvic magnetic resonance imaging (MRI) data of a patient;
operating a first deep learning segmentation model upon the pelvic MRI data to generate an initial localization of a plurality of target structures in the pelvic MRI data;
operating the first deep learning segmentation model to generate a probability map corresponding to the plurality of target structures;
generating enhanced imaging data using the initial localization, the probability map, and the pelvic MRI data;
operating a second deep learning segmentation model upon the enhanced imaging data to generate segmented imaging data identifying locations of the plurality of target structures; and
providing an output for a clinical workflow based upon the segmented imaging data.

9. The method of claim 8, wherein generating the enhanced imaging data comprises multiplying the pelvic MRI data by the probability map to generate a weighted image.

10. The method of claim 8, wherein a first target structure of the plurality of target structures is an anatomical structure and a second target structure of the plurality of target structures is a pathological region.

11. The method of claim 10, wherein the anatomical structure comprises an internal sphincter muscle or an external sphincter muscle, and the pathological region comprises a fistula tract.

12. The method of claim 8, further comprising generating a boundary box for a target structure of the plurality of target structures, and providing the boundary box as a prompt to the first deep learning segmentation model.

13. The method of claim 8, wherein the second deep learning segmentation model is a self-configuring segmentation model.

14. A non-transitory computer-readable medium storing computer-executable instructions that, when executed, cause a processor to perform operations, comprising:
accessing pelvic magnetic resonance imaging (MRI) data of a patient;
operating a first deep learning segmentation model upon the pelvic MRI data to generate an initial localization of a plurality of target structures in the pelvic MRI data;
operating the first deep learning segmentation model to generate a probability map corresponding to the plurality of target structures;
generating enhanced imaging data using the initial localization, the probability map, and the pelvic MRI data; and
operating a second deep learning segmentation model upon the enhanced imaging data to generate segmented imaging data identifying locations of the plurality of target structures.

15. The non-transitory computer-readable medium of claim 14, wherein generating the enhanced imaging data comprises multiplying the pelvic MRI data by the probability map to generate a weighted image.

16. The non-transitory computer-readable medium of claim 14, wherein a first target structure of the plurality of target structures is an anatomical structure and a second target structure of the plurality of target structures is a pathological region.

17. The non-transitory computer-readable medium of claim 14, wherein the second deep learning segmentation model is a self-configuring segmentation model.

18. The non-transitory computer-readable medium of claim 14, wherein the second deep learning segmentation model comprises a plurality of second deep learning segmentation models that are respectively configured to segment different target structures of the plurality of target structures.

19. The non-transitory computer-readable medium of claim 14, wherein the second deep learning segmentation model comprises a multi-class segmentation model configured to segment the plurality of target structures.

20. The non-transitory computer-readable medium of claim 14, wherein the operations further comprise evaluating the pelvic MRI data for imaging quality, excluding an image having an inaccurately centered field-of-view, noise, or artifact, and exporting the segmented imaging data as a DICOM segmentation object for storage in a picture archiving and communication system.
